(19) United States
(12) Patent Application Publication
(10) Pub. No.: US 2024/0366699 A1
(43) Pub. Date: Nov. 7, 2024

(54) YEAST FOR THE TREATMENT OF INFLAMMATION

(71) Applicant: Milmed Unico AB, Stockholm (SE)

(72) Inventors: Trevor ARCHER, Floda (SE); Rita BUSINARO, Rome (IT); Tomas LENZ, Örebro (SE)

(73) Assignee: Milmed Unico AB, Stockholm (SE)

(21) Appl. No.: 18/573,574

(22) PCT Filed: Jun. 29, 2022
(86) PCT No.: PCT/SE2022/050656

§ 371 Date: Dec. 22, 2023

(30) Foreign Application Priority Data
Jun. 29, 2021 (SE) 2150837-9

(51) Int. Cl. A61K 36/064; A61K 41/00; A61P 1/00; A61P 29/00; C12N 1/18; C12N 13/00

(52) U.S. Cl. CPC A61K 36/064; A61K 41/00; A61P 1/00; A61P 29/00; C12N 1/185; C12N 13/00

(57) ABSTRACT

A yeast cell for use in the treatment and/or alleviation of inflammation and/or symptoms caused by inflammation, wherein the yeast cell has been treated with electromagnetic waves in the range of 1 GHz to 300 GHz, or said yeast cell has been grown from a yeast cell treated with electromagnetic waves in the range of 1 GHz to 300 GHz, and composition comprising such a yeast cell.

### TECHNICAL FIELD

The present disclosure relates to a yeast cell for use in the treatment and/or alleviation of inflammation and/or symptoms caused by inflammation, as well as a composition comprising said yeast cell.

### BACKGROUND

Inflammation is the body's protective response against harmful stimuli such as pathogens, toxic compounds, damaged cells or irradiation. Chronic inflammation is a low, long-term inflammation lasting several months to years. Auto-inflammation occurs when the body produces an inflammatory response against its own tissues. The cardinal symptoms of inflammation are heat, pain, redness, swelling and loss of function.

Several different medicaments are usually used in the treatment of inflammation, many of which have side effects.

Thus, there is a need for new compositions and methods allowing for improved treatment or alleviation of inflammation and/or symptoms associated with inflammation.

An object of the present invention is to overcome these problems.

### SUMMARY

According to a first aspect, the above and other objects of the invention are achieved, in full or at least in part, by a yeast cell as defined by claim 1. According to this claim the above object is achieved by a yeast cell for use in the treatment and/or alleviation of inflammation and/or symptoms caused by inflammation, wherein the yeast cell has been treated with electromagnetic waves in the range of 1 GHz to 300 GHz, or said yeast cell has been grown from a yeast cell treated with electromagnetic waves in the range of 1 GHz to 300 GHz. The yeast cells may be grown from a yeast cell treated with electromagnetic waves in the range of 1 GHz to 300 GHz for several generations, such as up to 300, 200 or 100 generations, such as up to 95, 90, 85, 80, 75, 70, 65, 60, 55 or 50 generations, such as 47, 45, 42, 40, 37, 35, 32, 30, 27, 25, 22, 20, 17, 15, 12, 10, 7 or 5 generations.

An advantage with the yeast cell according to the present disclosure is that it allows for improved and cost-effective treatment and/or alleviation of inflammation and/or symptoms caused by inflammation.

In a second aspect, a composition for use in the treatment and/or alleviation of inflammation and/or symptoms caused by inflammation is provided, the composition comprising at least on yeast cell according to the present disclosure and an excipient and/or carrier.

Further advantageous features of the invention and its embodiments are defined in the appended claims and in the detailed description.

Other objectives, features and advantages of the present invention will appear from the following detailed disclosure, from the attached claims, as well as from the drawings. It is noted that the invention relates to all possible combinations of features.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of said element, device, component, means, step, etc., unless explicitly stated otherwise. The steps of any method disclosed herein do not have to be performed in the exact order disclosed, unless explicitly stated.

As used herein, the term “comprising” and variations of this term are not intended to exclude other additives, components, integers or steps.

### BRIEF DESCRIPTION OF THE DRAWINGS

Further objects, features and advantages will appear from the following detailed description, with reference being made to the accompanying drawings, in which:

FIG. 1A is a diagram showing a growth curve of untreated yeast cells.

FIG. 1B is a diagram showing a growth curve of treated cells.

FIGS. 2A and 2B are diagrams showing the effect of compositions according to the present disclosure on the LPS-induced expression of IL-10 (paired t-test: * p<0.05, ** p<0.01, *** p<0.005).

FIGS. 3A and 3B are diagrams showing the effect of compositions according to the present disclosure on the LPS-induced expression of IL-6 (paired t-test: * p<0.05, ** p<0.01, *** p<0.005).

FIGS. 4A and 4B are diagrams showing the effect of compositions according to the present disclosure on the LPS-induced expression of TNF-α (paired t-test: * p<0.05, ** p<0.01, *** p<0.005).

FIGS. 5A, 5B and 5C are diagrams showing the effect of compositions according to the present disclosure on the LPS-induced expression of iNOS (paired t-test: * p<0.05, ** p<0.01, *** p<0.005).

FIGS. 6A and 6B are diagrams showing the effect of compositions according to the present disclosure on the LPS-induced reduction of ARG-1-expression (paired t-test: * p<0.05, ** p<0.01, *** p<0.005).

FIG. 7 is a diagram showing the effect of compositions according to the present disclosure on the LPS-induced change in cell area (paired t-test: * p<0.05, ** p<0.01, *** p<0.005).

### DETAILED DESCRIPTION

The use of low-intensity electromagnetic millimeter waves within non-traditional areas, such as medicine, biology and biotechnology is a trend that originated in Russia in the middle of the 1960s.

It has now surprisingly been found that a yeast cell treated with electromagnetic waves in the range of 1 GHz to 300 GHz (a so called treated yeast cell or treated yeast) or a yeast cell grown from a treated yeast cell are effective in the treatment and/or alleviation of inflammation and/or symptoms caused by inflammation. An advantage with the yeast cell according to the present disclosure is that it allows for improved and cost-effective treatment and/or alleviation of inflammation and/or symptoms caused by inflammation. The yeast cell has not been genetically modified.

The yeast cells may have been grown from a yeast cell treated with electro-magnetic waves in the range of 1 GHz to 300 GHz for several generations, such as up to 300, 200 or 100 generations, such as up to 95, 90, 85, 80, 75, 70, 65, 60, 55 or 50 generations, such as 47, 45, 42, 40, 37, 35, 32, 30, 27, 25, 22, 20, 17, 15, 12, 10, 7 or 5 generations. Thus, the yeast cells may be treated with electromagnetic waves as described herein and thereafter grown for several generation, such as up to 300, 200 or 100 generations, such as up to 95, 90, 85, 80, 75, 70, 65, 60, 55 or 50 generations, such as 47, 45, 42, 40, 37, 35, 32, 30, 27, 25, 22, 20, 17, 15, 12, 10, 7 or 5 generations.

Typically, the yeast cells used in the applications described herein have been grown for 10 to 50, such as from 15 to 40, such as from 20 to 30, such as from 25 to 30, such as 26, 27, 28 or 29 generations. Preferably, the yeast cells used in the applications described herein may have been grown for 25 to 30 generations, more preferably for 27 generations.

The electromagnetic waves may be delivered with any electronic or photonic device known within the art. The electromagnetic waves may have a power density below 1 mW/cm², such as about 0.1 mW/cm², such as between 0.004 mW/cm² and 0.2 mW/cm².

According to one embodiment, the inflammation is selected from the group consisting of inflammatory bowel disease (IBD), irritable bowel syndrome (IBS); an autoimmune disease, and an inflammation caused by a microorganism; and/or wherein the symptom caused by inflammation is chosen from the group consisting of heat, pain, redness, swelling, loss of function and combinations thereof.

The inflammation may be selected from the group consisting of inflammatory bowel disease (IBD), irritable bowel syndrome (IBS).

The inflammatory bowel disease (IBD) may be Crohn's disease or ulcerative colitis.

The autoimmune disease may be multiple sclerosis, psoriasis, systemic lupus erythematosus (SLE) or polymyalgia reumatika.

The inflammation caused by a microorganism may be inflammation caused by shingles (herpes zoster) or lyme arthritis.

The inflammation caused by a microorganism may be chronic sinusitis.

According to another embodiment, the inflammation is inflammatory bowel disease (IBD), such as Crohn's disease or ulcerative colitis; or irritable bowel syndrome (IBS).

According to yet another embodiment, said electromagnetic waves are in the range from about 1 GHz to about 200 GHz, such as 10 to 100 GHz, such as 30 to 70 GHz, such as 40 to 65 GHz. The oscillation frequency may be within the range from about 35 to about 65 GHz. The oscillation frequency may be 42.2 GHz.

The electromagnetic waves may be delivered with any electronic or photonic device known within the art. The electromagnetic waves may have a power density below 1 mW/cm², such as between 0.004 mW/cm² and 0.2 mW/cm², such as about 0.1 mW/cm².

According to a further embodiment, said electromagnetic waves are chosen from group consisting of: 30 GHz, 31 GHz, 32 GHz, 33 GHz, 34 GHz, 35 GHz, 36 GHz, 37 GHz, 37.5 GHz, 38 GHz, 39 GHz, 40 GHz, 41 GHz, 42 GHz, 43 GHz, 44 GHz, 45 GHz, 46 GHz, 47 GHz, 48 GHz, 49 GHz, 50 GHz, 51 GHz, 52 GHz, 53 GHz, 54 GHz, 55 GHz, 56 GHz, 57 GHz, 58 GHz, 59 GHz, 60 GHz, 61 GHz, 62 GHz, 63 GHz, 64 GHz, 65 GHz, 66 GHz, 67 GHz, 68 GHz, 69 GHz, 70 GHz, 71 GHz, 72 GHz, 73 GHz, 74 GHz, 75 GHz, 76 GHz, 77 GHz, 78 GHz, 79 GHz, 80 GHz, 81 GHz, 82 GHz, 83 GHz, 84 GHz, 85 GHz, 86 GHz, 87 GHz, 88 GHz, 89 GHz and 90 GHz. Preferably, said electromagnetic waves are chosen from group consisting of: 37.5 GHz or 75 GHz. The skilled person realizes that the frequencies above are lifted/depressed to the closest complete number without decimal points. Thus, e.g. 40 GHz shall be understood as 40±0.5. The electromagnetic waves may be delivered with any electronic or photonic device known within the art. The electromagnetic waves may have a power density below 1 mW/cm², such as between 0.004 mW/cm² and 0.2 mW/cm², such as about 0.1 mW/cm².

In one specific embodiment, the oscillation frequency is 42194±10 NMz and linearly modulated within a 100 NMz band around this frequency. The electromagnetic waves may be delivered with any electronic or photonic device known within the art. The electromagnetic waves may have a power density below 1 mW/cm², such as between 0.004 mW/cm² and 0.2 mW/cm², such as about 0.1 mW/cm².

In another specific embodiment, the oscillation frequency is 53534±10 MHz and linearly modulated within a 50 MHz band around this frequency. The electromagnetic waves may be delivered with any electronic or photonic device known within the art. The electromagnetic waves may have a power density below 1 mW/cm², such as between 0.004 mW/cm² and 0.2 mW/cm², such as about 0.1 mW/cm².

In another specific embodiment, the oscillation frequency is 60124±10 MHz and linearly modulated within a 50 MHz band around this frequency. The electromagnetic waves may be delivered with any electronic or photonic device known within the art. The electromagnetic waves may have a power density below 1 mW/cm², such as between 0.004 mW/cm² and 0.2 mW/cm², such as about 0.1 mW/cm².

According to one embodiment, said electromagnetic waves have a power density below 1 mW/cm²; preferably said electromagnetic waves have a power density of between 0.004 mW/cm² and 0.2 mW/cm². Thus, the electromagnetic waves may have a power density of about 0.1 mW/cm².

According to another embodiment, said electromagnetic waves are modulated in a frequency within the range of 0.01% to about 0.5% of the average frequency.

According to a further embodiment, said yeast cell has been treated with electromagnetic waves for a time period of from 10 minutes to 240 minutes, such as from 20 minutes to 130 minutes, such as from 20 minutes to 120 minutes, such as from 30 minutes to 90 minutes, such as from 35 minutes to 85 minutes, such as 45 minutes to 80 minutes, such as 40 minutes, such as 50 minutes, such as 60 minutes, such as 70 minutes, such as 80 minutes. In some cases, the yeast cell has been treated with electromagnetic waves for a time period of 30 minutes to 50 minutes, more preferred for 40 minutes.

Preferably, the yeast cell has been treated with electromagnetic waves for a time period of 30 minutes to 110 minutes, such as 40 minutes to 105 minutes, such as 50 minutes to 100 minutes, such as 60 to 95 minutes, such as 70 to 90 minutes, such as 75 to 85 minutes, more preferred for 80 minutes.

According to a specific embodiment, said yeast cell has been treated with electromagnetic waves in the range from 30 GHz to 90 GHz, for a time period of from 20 minutes to 60 minutes.

Said yeast cell may have been treated with electromagnetic waves in the range from 35 GHz to 80 GHz, for a time period of from 30 minutes to 50 minutes.

Said yeast cell may have been treated with electromagnetic waves of 37.5 GHz, for a time period of from 30 minutes to 50 minutes, preferably 40 minutes.

Said yeast cell may have been treated with electromagnetic waves of 75 GHz, for a time period of from 30 minutes to 50 minutes, preferably 40 minutes.

According to a preferred specific embodiment, said yeast cell has been treated with electromagnetic waves in the range from 30 GHz to 90 GHz, for a time period of from 60 minutes to 100 minutes.

Said yeast cell may have been treated with electromagnetic waves in the range from 35 GHz to 80 GHz, for a time period of from 70 minutes to 90 minutes.

Said yeast cell may have been treated with electromagnetic waves of 37.5 GHz, for a time period of from 70 minutes to 90 minutes, preferably 80 minutes.

Said yeast cell may have been treated with electromagnetic waves of 75 GHz, for a time period of from 70 minutes to 90 minutes, preferably 80 minutes.

According to yet another embodiment, said yeast cell is of the genus Saccharomyces, such as a yeast cell being selected from the group consisting of Saccharomyces carlsbergensis or Saccharomyces cerevisiae. An advantage with this is that such yeast may be readily available at a low cost.

The yeast cell may be Saccharomyces cerevisiae S Ivovskaja-Milmed. This strain has been deposited as DSM 33148. The strain has previously been deposited as Y2483.

The yeast cell may be Saccharomyces carlsbergensis. This strain (treated as described herein) has been deposited as DSM 34143. Thus, the strain deposited as DSM 34143 is a treated yeast.

According to a second aspect of the present disclosure, a composition for use in the treatment and/or alleviation of inflammation and/or symptoms caused by inflammation is provided, the composition comprising at least one yeast cell according to the present disclosure and an excipient and/or a carrier. The composition may contain sterile wort, preferably 5 to 20 wt %, such as 8 to 15 wt %, such as 10 to 12 wt, such as 11 wt %. The composition may contain a carbohydrate, such as glucose and/or saccharose.

According to one embodiment, the composition further comprises at least one vitamin and/or at least one mineral. The vitamin may be chosen from the group consisting of A-vitamin, C-vitamin and D-vitamin or combinations thereof. The mineral may be chosen from the group consisting of zinc, magnesium and selenium and combinations thereof. The mineral may be in the form of a salt.

According to one embodiment, the composition is for oral intake. This is advantageous since it allows the patient to administer the composition him- or herself. Furthermore, oral intake gives a systemic effect via uptake in the gastrointestinal tract. The composition for oral intake may be a suspension.

The treated yeast may be distributed to the subject in any form suitable, such as a liquid, a powder, a gel or as pill.

A single dose may comprise 10×10⁶ CFU/dose to 20,000×10⁶ CFU/dose, such as 100×10⁶ CFU/dose to 17,000×10⁶ CFU/dose, such as 200×10⁶ CFU/dose to 15,000×10⁶ CFU/dose, such as 300×10⁶ CFU/dose to 12,000×10⁶ CFU/dose, such as 500×10⁶ CFU/dose to 10,000×10⁶ CFU/dose, such as 500×10⁶ CFU/dose to 8,000×10⁶ CFU/dose, such as 1,000×10⁶ CFU/dose to 6,000×10⁶ CFU/dose, such as 2,000×10⁶ CFU/dose to 5,000×10⁶ CFU/dose, such as 3,000×10⁶ CFU/dose to 4,000×10⁶ CFU/dose. CFU=colony forming unit.

Preferably, a single dose for humans comprises from 100×10⁶ CFU/dose to 6,000×10⁶ CFU/dose, such as 300×10⁶ CFU/dose to 3,000×10⁶ CFU/dose, such as 600×10⁶ CFU/dose to 1,000×10⁶ CFU/dose.

Preferably, a single dose for a larger mammal, such as a horse or cow, comprises from 6,000×10⁶ CFU/dose to 20,000×10⁶ CFU/dose, such as 8,000×10⁶ CFU/dose to 18,000×10⁶ CFU/dose, such as 10,000×10⁶ CFU/dose to 15,000×10⁶ CFU/dose, such as 12,000×10⁶ CFU/dose.

Preferably, a single dose for a smaller mammal, such as a rabbit, cat or dog, comprises 10×10⁶ CFUs/dose to 1,500×10⁶ CFUs/dose, such as 100×10⁶ CFU/dose to 1,000×10⁶ CFU/dose, such as 200×10⁶ CFU/dose to 800×10⁶ CFU/dose, such as 300×10⁶ CFU/dose to 600×10⁶ CFU/dose, such as 500×10⁶ CFU/dose.

Yeast cells according to the present disclosure may be administered in an amount of from 50×10⁶ CFUs/day to 10,000×10⁶ CFUs/day, such as 100×10⁶ CFUs/day, such as 100×10⁶ CFUs/day to 8,000×10⁶ CFUs/day, such as 500×10⁶ CFUs/day to 5,000×10⁶ CFUs/day, such as 700×10⁶ CFUs/day to 4,000×10⁶ CFUs/day, such as 800×10⁶ CFUs/day to 3,000×10⁶ CFUs/day, such as 900×10⁶ CFUs/day to 2,000×10⁶ CFUs/day, such as 1,000×10⁶ CFUs/day to 1,500×10⁶ CFUs/day.

Yeast cells according to the present disclosure may be administered to a human in an amount of from 500×10⁶ CFUs/day to 1,500×10⁶ CFUs/day, such as 750×10⁶ CFUs/day to 1,500×10⁶ CFUs/day, such as 800×10⁶ CFU/day to 1,200×10⁶ CFU/day, such as 900×10⁶ CFUs/day.

Yeast cells according to the present disclosure may be administered to a larger mammal, such as a horse or cow, in an amount of from 1,000×10⁶ CFUs/day to 10,000×10⁶ CFUs/day, such as 2,000×10⁶ CFUs/day to 8,000×10⁶ CFUs/day, such as 3,000×10⁶ CFUs/day to 6,000×10⁶ CFUs/day, such as 4,000×10⁶ CFUs/day to 5,000×10⁶ CFUs/day.

Yeast cells according to the present disclosure may be administered a smaller mammal, such as a rabbit, cat or dog, in an amount of from 10×10⁶ CFUs/day to 2,000×10⁶ CFUs/day, such as 50×10⁶ CFUs/day to 1,500×10⁶ CFUs/day, such as 100×10⁶ CFUs/day to 1,000×10⁶ CFUs/day, such as 200×10⁶ CFUs/day to 800×10⁶ CFUs/day, such as 300×10⁶ CFUs/day to 600×10⁶ CFUs/day, such as 400×10⁶ CFUs/day to 500×10⁶ CFUs/day.

Yeast cells according to the present disclosure may be administered in an amount of from 200×10⁶ CFUs/week to 70,000×10⁶ CFUs/week, such as 300×10⁶ CFUs/week to 50,000×10⁶ CFUs/week, such as 500×10⁶ CFUs/week to 25,000×10⁶ CFUs/week, such as 1,000×10⁶ CFUs/week to 20,000×10⁶ CFUs/week, such as 1,500×10⁶ CFUs/week to 15,000×10⁶ CFUs/week, such as 3,000×10⁶ CFUs/week to 10,000×10⁶ CFUs/week, such as 5,000×10⁶ CFUs/week to 7,500×10⁶ CFUs/week, such as 6,000×10⁶ CFUs/week.

Yeast cells according to the present disclosure may be administered to a human in an amount of from 3,500×10⁶ CFUs/week to 9,000×10⁶ CFUs/week, such as from 4,500×10⁶ CFUs/week to 8,000×10⁶ CFUs/week, such as 5,000×10⁶ CFUs/week to 7,000×10⁶ CFUs/week, such as 6,000×10⁶ CFUs/week.

Yeast cells according to the present disclosure may be administered to a larger mammal, such as a horse or cow, in an amount of from 7,000×10⁶ CFUs/week to 70,000×10⁶ CFUs/week, such as 10,000×10⁶ CFUs/week to 50,000×10⁶ CFUs/week, such as 15,000×10⁶ CFUs/week to 40,000×10⁶ CFUs/week, such as 20,000×10⁶ CFUs/week to 30,000×10⁶ CFUs/week, such as 22,000×10⁶ CFUs/week to 27,000×10⁶ CFUs/week, such as 24,000×10⁶ CFUs/week to 26,000×10⁶ CFUs/week, such as 25,000×10⁶ CFUs/week.

Yeast cells according to the present disclosure may be administered a smaller mammal, such as a rabbit, cat or dog, in an amount of from 70×10⁶ CFUs/week to 14,000×10⁶ CFUs/week, such as 100×10⁶ CFUs/week to 10,000×10⁶ CFUs/week, such as 300×10⁶ CFUs/week to 6,000×10⁶ CFUs/week, such as 500×10⁶ CFUs/week to 4,000×10⁶ CFUs/week, such as 700×10⁶ CFUs/week to 3,000×10⁶ CFUs/week, such as 1,000×10⁶ CFUs/week to 2,000×10⁶ CFUs/week.

The time period for treatment may be at least two weeks, at least three weeks, at least four weeks, at least five weeks, at least six weeks, at least seven week, at least eight week, at least nine week, at least ten weeks, at least 15 weeks, or at least 20 weeks. In certain cases the treatment may be longer than 20 weeks.

According to one embodiment, the composition is a liquid composition.

In one specific embodiment, the composition is in the form of a malt beverage or in any kind of beverage comprising the treated yeast or yeast cells grow from treated yeast.

According to one embodiment, the liquid composition comprises the yeast cell in an amount of from 10×10⁶ CFUs/ml to 50×10⁶ CFUs/ml, preferably from 20×10⁶ CFUs/ml to 40×10⁶ CFUs/ml, such as 30×10⁶ CFUs/ml. Such a composition may be administered one, two, three, four, five, six or seven times a week, preferably two or three times a week. In one preferred embodiment a total 200 ml of a composition comprising 30×10⁶ CFUs/ml is administered per week, divided into two or three portions. In another preferred embodiment a total 200 ml of a composition comprising 40×10⁶ CFUs/ml is administered per week, divided into two or three portions.

According to one embodiment, the composition is a powder composition.

In certain cases, the powder composition may comprise viable, dried yeast. The dried yeast may be prepared from a suspension of yeast cells in wort, such as 5 to 20 wt %, such as 8 to 15 wt %, such as 10 to 12 wt, such as 11 wt %. Drying of yeast is a standard procedure well known to the skilled person.

In certain cases, the powder composition may comprise viable, freeze-dried treated yeast. The freeze-dried yeast may be prepared from a suspension of yeast cells in wort, such as 5 to 20 wt %, such as 8 to 15 wt %, such as 10 to 12 wt, such as 11 wt %. Freeze-drying of yeast is a standard procedure well known to the skilled person.

In other cases, the powder composition may comprise viable, spray-dried treated yeast. The spray-dried yeast may be prepared from a suspension of yeast cells in wort, such as 5 to 20 wt %, such as 8 to 15 wt %, such as 10 to 12 wt, such as 11 wt %. Spray-drying of yeast is a standard procedure well known to the skilled person.

Alternatively, the powder composition may comprise viable treated yeast that has been dried in sterile warm air. Drying in sterile warm air is a gentle way of drying the yeast cells. The yeast dried in sterile warm air may be prepared from a suspension of yeast cells in wort, such as 5 to 20 wt %, such as 8 to 15 wt %, such as 10 to 12 wt, such as 11 wt %. To protect the yeast cells during the drying process, an emulsifier has been added to the yeast cells before drying in warm air.

The powder composition may comprise carbohydrates, such as glucose and/or saccharose.

Preferably, the powder composition comprises 2×10¹⁰ CFUs/mg to 4×10¹⁰ CFUs/mg, such as 2.5×10¹⁰ CFUs/mg to 3.5×10¹⁰ CFUs/mg, such as 2.7×10¹⁰ CFUs/mg to 3.2×10¹⁰ CFUs/mg, such as 2.8×10¹⁰ CFUs/mg to 3.1×10¹⁰ CFUs/mg, such as 2.9×10¹⁰ CFUs/mg to 3.0×10¹⁰ CFUs/mg.

Preferably, the powder composition has a moisture content of less than 10 wt %, such as less than 7.5 wt %, such as less than 5 wt %, such as less than 4 wt %, such as less than 3.9 wt %, such as less than 3.8 wt %, such as 3.7 wt %.

The powder composition may be administered one, two, three, four, five, six or seven times a week. Preferably, the powder composition is administered two to four, such as three times, per week. Alternatively, the powder composition may be administered one, two, three or four times a day, preferably two to three times a day.

The powder composition may be suspended in a liquid, such as water or aqueous beverage, such as a juice, before intake. The liquid may comprise carbohydrates, such as glucose and/or saccharose. When the powder composition comprises carbohydrates, such as glucose and/or saccharose, the powder composition may be suspended in water. The powder composition is preferably suspended to a concentration of from 10×10⁶ CFUs/ml to 50×10⁶ CFUs/ml, preferably from 20×10⁶ CFUs/ml to 40×10⁶ CFUs/ml, such as 30×10⁶ CFUs/ml.

The powder composition may be formulated as a capsule or tablet for oral intake. Preferably, such a capsule or tablet comprises from 300×10⁶ CFU/capsule or tablet to 7,500×10⁶ CFU/capsule or tablet, such as 400×10⁶ CFU/capsule or tablet to 7,000×10⁶ CFU/capsule or tablet, such as 500×10⁶ CFU/capsule or tablet to 6,500×10⁶ CFU/capsule or tablet, such as 750×10⁶ CFU/capsule or tablet to 6,000×10⁶ CFU/capsule or tablet, such as 1,000×10⁶ CFU/capsule or tablet to 5,000×10⁶ CFU/capsule or tablet, such as 2,000×10⁶ CFU/capsule or tablet to 4,000×10⁶ CFU/capsule or tablet, such as 2,500×10⁶ CFU/capsule or tablet to 3,500×10⁶ CFU/capsule or tablet, such as 3,000×10⁶ CFU/capsule or tablet.

Such a capsule or tablet may be administered one, two, three, four, five, six or seven times a week, preferably two or three times a week.

Such a capsule or tablet for intake two or three times a week preferably comprises from 1,000×10⁶ CFU/capsule or tablet to 5,000×10⁶ CFU/capsule or tablet, such as 2,000×10⁶ CFU/capsule or tablet to 4,000×10⁶ CFU/capsule or tablet, such as 3,000×10⁶ CFU/capsule or tablet. More preferably, such a capsule or tablet for intake two or three times a week comprises 4,000×10⁶ CFU/capsule or tablet.

Alternatively, one capsule or tablet may be administrated orally once a day. Such a capsule or tablet for daily intake preferably comprises from comprising 100×10⁶ CFUs to 3,000×10⁶ CFUs, such as 200×10⁶ CFUs to 2,500×10⁶ CFUs, such as 300×10⁶ CFUs to 2,000×10⁶ CFUs, 500×10⁶ CFUs to 1,500×10⁶ CFUs, such as 750×10⁶ CFUs to 1,500×10⁶ CFUs, such as 800×10⁶ CFU/capsule to 1,200×10⁶ CFU/capsule, such as 900×10⁶ CFUs, is administered per day.

The composition according to the present disclosure is obtainable by a method comprising the steps: preparing a growth medium; sterilizing or pasteurizing the growth medium; growing yeast cells in the growth medium; and treating the yeast with electromagnetic waves, wherein the electromagnetic waves are in the range of 30 GHz to 300 GHz. The electromagnetic waves are preferably within the range from about 1 to 300 GHz, such as 1 to 200 GHz, more preferably 10 to 100 GHz, such as 20 to 80 GHz such as 30 to 70 GHz, such as 35 to about 65 GHz, such as 40 to 65 GHz, such as 30 GHz, 31 GHz, 32 GHz, 33 GHz, 34 GHz, 35 GHz, 36 GHz, 37 GHz, 37.5 GHz, 38 GHz, 39 GHz, 40 GHz, 41 GHz, 42 GHz, 43 GHz, 44 GHz, 45 GHz, 46 GHz, 47 GHz, 48 GHz, 49 GHz, 50 GHz, 51 GHz, 52 GHz, 53 GHz, 54 GHz, 55 GHz, 56 GHz, 7 GHz, 58 GHz, 59 GHz, 60 GHz, 61 GHz, 62 GHz, 63 GHz, 64 GHz, 65 GHz, 66 GHz, 67 GHz, 68 GHz, 69 GHz, 70 GHz, 71 GHz, 72 GHz, 73 GHz, 74 GHz, 75 GHz, 76 GHz, 77 GHz, 78 GHz, 79 GHz, 80 GHz, 81 GHz, 82 GHz, 83 GHz, 84 GHz, 85 GHz, 86 GHz, 87 GHz, 88 GHz, 89 GHz and 90 GHz.

Preferably, said electromagnetic waves are chosen from group consisting of: 37.5 GHz or 75 GHz.

Preferably, the oscillation frequency is 42194±10 MHz and linearly modulated within 100 MHz band around this frequency.

In one preferred embodiment, the oscillation frequency is 42194±10 MHz and linearly modulated within a 100 MHz band around this frequency.

In another preferred embodiment, the oscillation frequency is 53534±10 MHz and linearly modulated within a 50 MHz band around this frequency.

In another preferred embodiment the oscillation frequency is 60124±10 MHz and linearly modulated within a 50 MHz band around this frequency.

The electromagnetic waves may be delivered with any electronic or photonic device known within the art, such as a YAV-1 therapeutic device, based on an IMPATT diode oscillator.

The electromagnetic waves may have a power density below 1 mW/cm², such as between 0.004 mW/cm² and 0.2 mW/cm², e.g. about 0.1 mW/cm².

The present disclosure also provides a method for treating and/or alleviating inflammation and/or symptoms caused by inflammation, wherein the method comprises administering, to a subject in need thereof, a yeast cell that has been treated with electromagnetic waves in the range of 1 GHz to 300 GHz, or a yeast cell that has been grown from a yeast cell treated with electromagnetic waves in the range of 1 GHz to 300 GHz as explained above.

The subject may be any mammal, such as e.g. a human. Further, the mammal may be a domestic mammal, such as a horse, a cow, a camel, a cat or a dog. EHF energy is thus transferred into the treated subject in form of treated yeast externally stimulated by EHF radiation.

The invention can be implemented in any suitable form including food products, feed, other drink products, etc., or any combination of these, without departing from the gist of the invention.

### EHF-Treatment

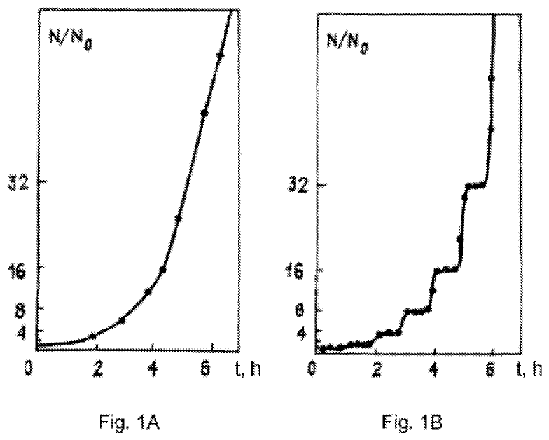

The effect of the EHF (extremely high frequency) treatment is shown in FIG. 1 (also described in WO 2011/023769).

FIG. 1A is a growth curve of untreated cells. N/No (Y axis) is the ratio of the number of cells N in the culture to the starting number No and t (in hours, X axis) is the culture-development time. FIG. 1B is a growth curve of treated cells. The frequencies of the oscillations generated by the cells can be synchronized by corresponding reorganization of the information structures of the cells, which causes differences in the division-cycle durations of individual cells to be practically eliminated with the result of “steps” on the growth curve. It is apparent from FIG. 1B that after each division cycle the number of cells is doubled synchronously, so that the dependence of the number of cells on time is represented by a step curve.

Table 1 is an overview of the minimum time (to, min) needed to synchronize cell division of all cells at different power density levels (P, mW/cm²) with a radiation frequency 42.2 GHz.

TABLE 1
The minimum time (t₀, min) needed to synchronize cell
division of all cells at different power density levels (P,
mW/cm²) with a radiation frequency 42.2 GHz.

| t₀, min | P, mW/cm² |
|---|---|
| 126 | 0.005 |
| 103 | 0.009 |
| 81 | 0.015 |
| 60 | 0.026 |
| 49 | 0.040 |
| 38 | 0.077 |
| 36 | 0.130 |
| 34 | 0.209 |

Table 2 is an overview of time required (to, min) to synchronize cell division of 15 percent of the cells at different power density levels (P, mW/cm²) with a radiation frequency of 42.2 GHz.

TABLE 2
Time required (t₀, min) to synchronize cell division of
15 per cent of the cells at different power density levels (P,
mW/cm²) with a radiation frequency of 42.2 GHz

| t₀, min | P, mW/cm² |
|---|---|
| 111 | 0.003 |
| 86 | 0.006 |
| 65 | 0.012 |
| 45 | 0.024 |
| 38 | 0.037 |
| 33 | 0.052 |
| 31 | 0.074 |
| 27 | 0.130 |
| 26 | 0.200 |

Thus, a preferred EHF treatment time is between 20 and 120 minutes.

The method may further comprise the step of growing the treated yeast cells in the growth medium. The growth may be aborted at any time, when a desired cell concentration is achieved.

The growth medium may be wort, i.e. a tonic malt beverage obtained from wort and yeast. Any kind of yeast may be used. Any kind of wort may be used. Alternatively, the wort is obtained from a brewery or is made from barley malt or made from wort concentrates.

The wort may be pasteurized such as by heating it to between 7° and 75° C. for more than 30 minutes. The wort may then be stored in sealed containers up to two weeks at temperatures between 18 and 20° C.

S. cerevisiae may be revived by suspension in a small volume of sterilized 11 wt % wort. It is important that no other microorganisms contaminate the wort.

The revived culture is subsequently inoculated on a number of Petri dishes with agarized wort, to obtain pure yeast culture. This may be confirmed by microscope.

Prior to EHF-treatment, yeast from one of the dishes with sterile pure culture are transferred into a tube containing sterile 11 wt % wort, such as between 5 and 50 mL, such as between 6 and 40 mL, such as between 7 and 30 mL, such as between 8 and 20 mL, such as between 9 and 15 mL, such as between 10 to 12 mL. The cultures are grown until skim appears, typically at 25 to 28° C. during 20 to 24 hours.

The yeast culture is then treated in an EHF-field. This may be done by first filling sterile Petri dishes with yeast suspension. The dish is then covered and placed in an EHF-unit. Such a unit may be any unit generating electromagnetic oscillations in the EHF-range. EHF-treating time is less than 240 minutes, such as less than 130 minutes, such as less than 120 minutes, such as less than 110 minutes, such as less than 100 minutes, preferably less than 90 minutes, such as 85 minutes, such as 80 minutes, such as 70 minutes, such as 60 minutes, such as 50 minutes, such as 40 minutes. The power density of EHF-oscillations is preferably about 0.1 mW/cm². The oscillation frequency is within the range of 30 to 300 GHz. The electromagnetic waves may be within the range from about 30 to 90 GHz, such as 35 to about 65 GHz, such as 30 GHz, 31 GHz, 32 GHz, 33 GHz, 34 GHz, 35 GHz, 36 GHz, 37 GHz, 37.5 GHz, 38 GHz, 39 GHz, 40 GHz, 41 GHz, 42 GHz, 43 GHz, 44 GHz, 45 GHz, 46 GHz, 47 GHz, 48 GHz, 49 GHz, 50 GHz, 51 GHz, 52 GHz, 53 GHz, 54 GHz, 55 GHz, 56 GHz, 57 GHz, 58 GHz, 59 GHz, 60 GHz, 61 GHz, 62 GHz, 63 GHz, 64 GHz, 65 GHz, 66 GHz, 67 GHz, 68 GHz, 69 GHz, 70 GHz, 71 GHz, 72 GHz, 73 GHz, 74 GHz, 75 GHz, 76 GHz, 77 GHz, 78 GHz, 79 GHz, 80 GHz, 81 GHz, 82 GHz, 83 GHz, 84 GHz, 85 GHz, 86 GHz, 87 GHz, 88 GHz, 89 GHz or 90 GHz. Preferably, said electromagnetic waves are chosen from group consisting of: 37.5 GHz or 75 GHz.

In a specific embodiment, the oscillation frequency is 42194±10 MHz and linearly modulated within a 100 MHz band around this frequency.

In another specific embodiment, the oscillation frequency is 53534±10 MHz and linearly modulated within a 50 MHz band around this frequency.

In yet another specific embodiment, the oscillation frequency is 60124±10 MHz and linearly modulated within a 50 MHz band around this frequency.

The electromagnetic waves may be delivered with any electronic or photonic device known within the art, such as a YAV-1 therapeutic device, based on an IMPATT diode oscillator.

The frequency modulation of the electromagnetic waves may be from 0% to about 0.5% of the respective average frequency, such as 0.5% of the respective average frequency.

After treatment in the EHF-unit, the abovementioned treated suspension is transferred to a tube, such as a 50 to 100 mL tube, containing sterile 11 wt % wort. The cells are allowed to grow until skim appears, typically during 20 to 24 hours at 25 to 28° C. This is the seed material.

The seed material is then added to pasteurized or sterilized wort, typically 2 to 3 L, filled in containers (tube, can, etc.) of nominal capacity slightly larger than the amount word, typically 4 to 5 L, and cultivated until a cell concentration of 30×10⁶ cells/mL is achieved, typically after 20 to 24 hours at 25 to 28° C.

If large volumes of beverage are produced, the above-mentioned treatment may be implemented in several stages by adding the result of a previous cultivation cycle as seeding material to sterile wort with a ration of 1:10 seeding material:wort. The cells are allowed to grow until skim appears, typically during 20 to 24 hours at 25 to 28° C. The last stage of the beverage production stage is deemed to be finished when a cell concentration no less than 30 million cells/mL is achieved.

Upon completion of the production stage, the beverage is ready for consumption and may be transferred to suitable transport vessels, e.g. bottles or cans. If storage is required, the beverage may be cooled to about 2 to 6° C., such as 2 to 4° C. and may then be stored, such as up to three weeks.

Saccharomyces carlsbergensis may be treated in the same manner as described above.

### Treated Yeast Cells

The following is an enabling embodiment of a production procedure. However, many different alternate production procedures are possible, which will be recognized by a person skilled in the art.

Wort was obtained from a brewery and the weight fraction of dry matter in the diluted wort was adjusted to 11 wt % (11 wt % wort).

The wort was sterilized in an autoclave chamber with a pressure of 0.05 MPa during 20 minutes and stored between 18 and 20° C.

Yeast, S. cerevisiae (DSM 33148) was revived by suspension in a small volume of sterilized 11 wt % wort under sterile conditions.

The yeast was inoculated on a number of Petri dishes with agarized wort, to obtain pure yeast culture. This was confirmed by microscope.

Prior to EHF-treatment, yeast from one of the dishes with pure culture sterile was transferred into the tube containing 11 mL of sterile 11 wt % wort. The cultures were grown at 28° C. during 20 to 24 hours until skim appeared.

The yeast culture was then treated in an EHF-field. This was done by first filling sterile Petri dishes with yeast suspension. The dish was then covered and placed in an EHF-unit, generating electromagnetic oscillations in the EHF-range. EHF-treating time was 80 minutes. The power density of EHF-oscillations was kept near 0.1 mW/cm². The oscillation frequency was 53534±10 MHz and was linearly modulated within a 50 MHz band around this frequency. The electromagnetic radiation was generated by a YAV-1 therapeutic device, based on an IMPATT diode oscillator.

After treatment in the EHF-unit, the abovementioned treated suspension was transferred to a tube of 75 mL containing sterile 11 wt % wort. The cells were allowed to grow during 22 hours at 28° C. until skim appears. This was the seed material.

The seed material was then added to 3 L pasteurized or sterilized wort filled in tubes of nominal capacity of 5 L and cultivated until a cell concentration of 30×10⁶ CFUs/mL was achieved. These treated yeast cells were used in the studies described below.

The treated yeast cells may alternatively be freeze dried and formulated into capsules. Such capsules were used in the patient study described below.

### Experimental Data

Compositions according to the present disclosure were evaluated in an in vitro model of inflammation. In summary, compositions according to the present disclosure (i.e. compositions comprising treated yeast cells according to the present disclosure) show an anti-inflammatory effect by reducing the expression the pro-inflammatory markers IL-1β, Il-6, TNF-α, and iNOS, which are induced by LPS. Furthermore, compositions according to the present disclosure (i.e. compositions comprising treated yeast cells according to the present disclosure) show an anti-inflammatory effect by increasing the expression of the anti-inflammatory marker ARG-1.

### Material

Dulbecco's Modified Eagle Medium (DMEM) High Glucose, Aurogene; Fetal Bovine Serum (FBS), Corning; Lipopolysaccharides (LPS) from Escherichia coli O111:B4, Sigma; Dulbecco's Phosphate Buffered Saline w/o Calcium, w/o Magnesium, Aurogene; QIAzol Lysis Reagent 50 ml, Qiagen; Well Cell Culture Cluster Flat Bottom with Lid Tissue Culture Treated, Corning.

### Method

Composition A: treated yeast cells in wort, supplemented with 2% (weight/volume) glucose.

Composition B: dried treated yeast cells in yeast extract peptone dextrose (YEPD), supplemented with 2% (weight/volume) glucose.

Untreated cells: dried untreated yeast cells in yeast extract peptone dextrose (YEPD), supplemented with 2% (weight/volume) glucose.

Day 1: 1,000,000 BV2 cells (a type of microglial cell derived from C57/BL6 murine) grown in DMEM High Glucose medium+5% FBS were plated in 6 well plates (1,000,000/ml cells in each well −14 conditions-). Each well contained 1 mL medium. The plates were placed at 37° C. over night.

Day 2: A composition according to present disclosure were added to the BV-2 cells at a concentration of 5×10² CFUs per well and incubated at 370 for 45 minutes. In control experiments, the BV-cells were and incubated at 37° for 45 minutes. After this pretreatment, lipopolysaccharide (LPS) (50 ng/mL PBS) was added to a final concentration of 1 ng/ml and the cells were incubated for 3 h and 4 h at 37° C. After 4 h, the supernatant was recovered in autoclaved 1.5 ml Eppendorf tubes, centrifuged at 12,000 rpm for 5 min and the pellet was removed, and then stored at −80° C. The adherent cells were also recovered by adding 700 ul of Qiazol and stored at −80° C. until RNA extraction, which was reverse transcribed and analysed by real-time RT-PCR.

### Analysis

The mRNA expression of the pro-inflammatory markers IL-1β, Il-6, TNF-α, and iNOS as well as the anti-inflammatory marker ARG-1 were analysed by RT-PCR. IL-1β is a strong pro-inflammatory cytokine that is upregulated in response to LPS. Its function is to fight infection. IL-6 is a strong pro-inflammatory cytokine (from lymph tissue) but also an anti-inflammatory myokine (from muscle tissue). IL-6 is upregulated in response to LPS. TNF-α is a strong pro-inflammatory cytokine with the same function as IL-1β and is also upregulated in response to LPS. iNOS (inducible nitric oxide synthase) catalyses production of nitric oxide (NO), involved in body defense mechanisms against pathogens, thereby inducing inflammation. iNOS is upregulated in response to LPS. ARG-1 is the gene encoding arginase, involved in immunologic crossreactivity, i.e. the extent to which different antigens appear similar to the immune system.

Cell areas (after exposure to compositions corresponding to “composition B” and “Untreated cells”) were calculated from actin immunofluorescence experiments taking advantage of the computerized program of Apotome microscope.

### Results

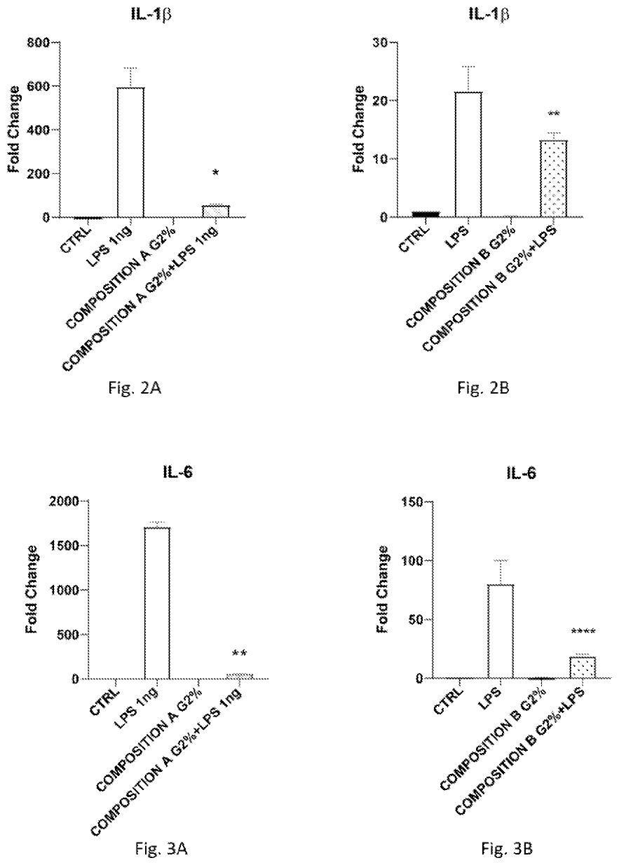

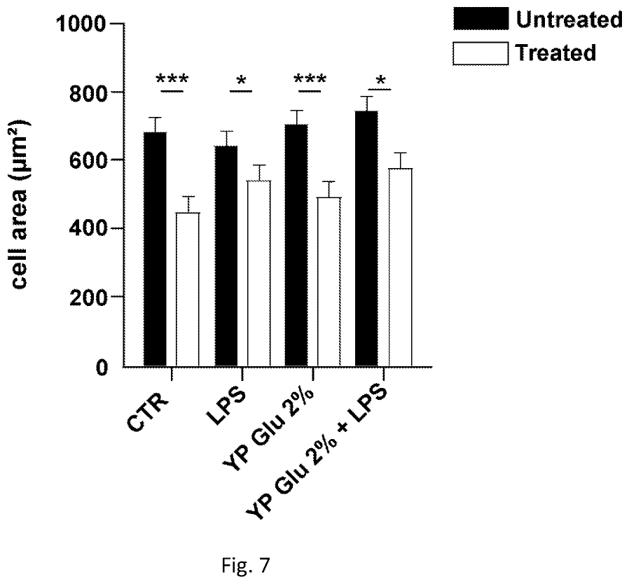

The results presented in FIGS. 2 to 6 are the results from a study using Saccharomyces cerevisiae (deposited as DSM 33148) and treated as described above. The results presented in FIG. 7 are the results from a study using Saccharomyces carlsbergensis (deposited as DSM 34143).

In FIGS. 2 to 7: paired t-test: * p<0.05, ** p<0.01, *** p<0.005.

Expression of IL-1β: As can be seen in FIGS. 2a and 2b, the mRNA expression of IL-1β is upregulated in cells exposed to LPS. IL-10 is not upregulated after treatment with compositions according to the present disclosure. Importantly, the compositions according to the present disclosure have an anti-inflammatory effect, since the mRNA expression of IL-10 is significantly decreased by combined treatment with LPS and compositions according to the present disclosure as compared to treatment with LPS alone. Thus, compositions according to the present disclosure block the effect of pro-inflammatory LPS. Untreated yeast cell (i.e. yeast cells that had not been EHF-treated as described above) blocked the upregulation of IL-10 caused by LPS to a lesser extent than treated yeast (data not shown).

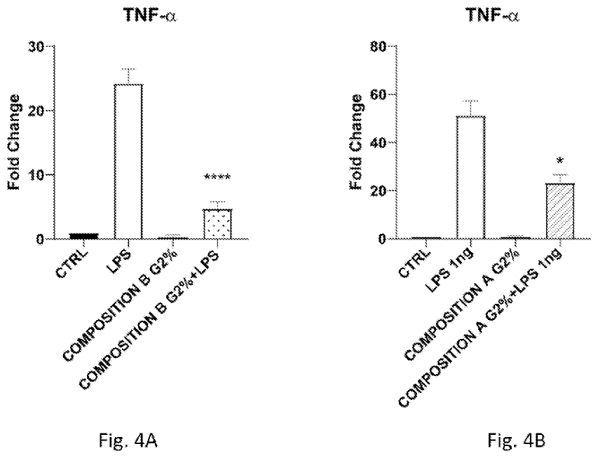

Expression of IL-6: As can be seen in FIGS. 3a and 3b, the mRNA expression of IL-6 is upregulated in cells exposed to LPS. IL-6 is not upregulated after treatment with compositions according to the present disclosure. Importantly, the compositions according to the present disclosure have an anti-inflammatory effect, since the mRNA expression of IL-6 is significantly decreased by combined treatment with LPS and compositions according to the present disclosure as compared to treatment with LPS alone. Thus, compositions according to the present disclosure block the effect of pro-inflammatory LPS. Untreated yeast cell (i.e. yeast cells that had not been EHF-treated as described above) blocked the upregulation of IL-6 caused by LPS to a lesser extent than treated yeast (data not shown).

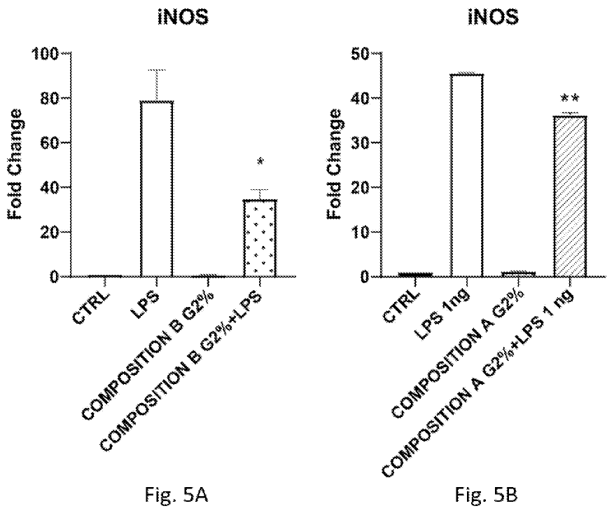

Expression of TNF-α: As can be seen in FIGS. 4a and 4b, the mRNA expression of TNF-α is upregulated in cells exposed to LPS. TNF-α is not upregulated after treatment with compositions according to the present disclosure. Importantly, the compositions according to the present disclosure have an anti-inflammatory effect, since the mRNA expression of TNF-α is significantly decreased by combined treatment with LPS and compositions according to the present disclosure as compared to treatment with LPS alone. Thus, compositions according to the present disclosure block the effect of pro-inflammatory LPS. Untreated yeast cell (i.e. yeast cells that had not been EHF-treated as described above) blocked the upregulation of TL-6 caused by LPS to a lesser extent than treated yeast (data not shown).

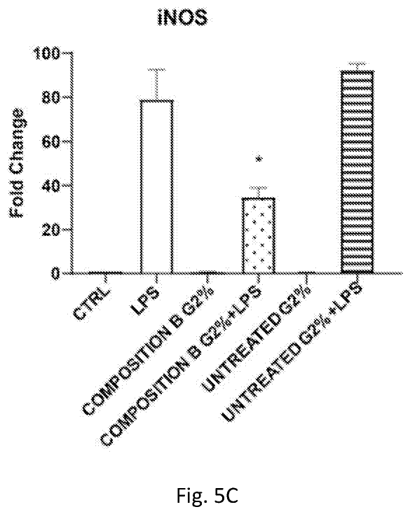

Expression of iNOS: As can be seen in FIGS. 5a and 5b, the mRNA expression of iNOS is upregulated in cells exposed to LPS. iNOS is not upregulated after treatment with compositions according to the present disclosure. Importantly, the compositions according to the present disclosure have an anti-inflammatory effect, since the mRNA expression of iNOS is significantly decreased by combined treatment with LPS and compositions according to the present disclosure as compared to treatment with LPS alone. Thus, compositions according to the present disclosure block the inflammatory NO production, an effect of pro-inflammatory LPS. In addition to the data presented in FIG. 5a, FIG. 5c also shows that iNOS is upregulated in cells exposed to LPS after treatment with compositions comprising untreated yeast cells. Thus, the anti-inflammatory effect is only demonstrated for compositions according to the present disclosure (EHF-treated yeast as described above). In other words, the blockade of the proinflammatory effect of LPS on the BV2 cells was only seen for compositions according to the present disclosure, but not for the untreated yeast composition.

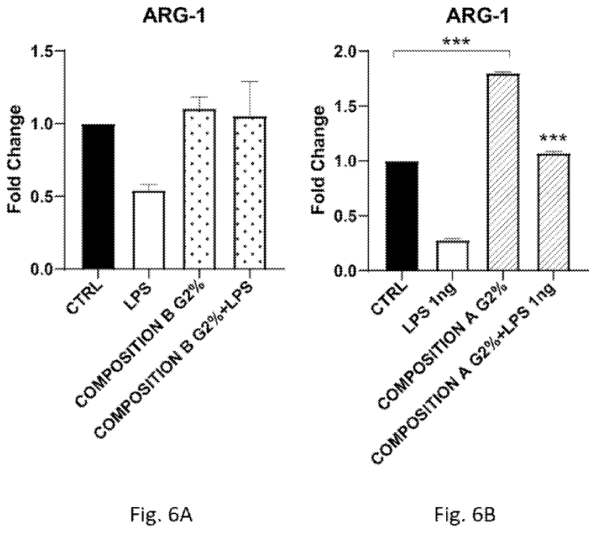

Expression of ARG-1: As can be seen in FIGS. 6a and 6b, the mRNA expression of ARG-1 is downregulated in cells exposed to LPS. ARG-1 is slightly upregulated after treatment with compositions according to the present disclosure. Importantly, the compositions according to the present disclosure have a pro-inflammatory effect, since the mRNA expression of ARG-1 is significantly increased by combined treatment with LPS and compositions according to the present disclosure as compared to treatment with LPS alone. The compositions according to the present disclosure induced direct anti-inflammatory effects, i.e. not related to the anti-proinflammatory effect of LPS. Thus, compositions according to the present disclosure influence the cells towards an anti-inflammatory phenotype.

Cell size: BV2 cells generally vary from about 10 to 15 Lim in diameter (corresponding to an area of about 80 to 180 μm²), are uniformly round, and exhibit occasional thin cytoplasmic projections (data not shown). Following treatment with lipopolysaccharide (LPS) with concurrent LPS activation, the BV2 cells are generally larger in diameter (25 to 30 μm (corresponding to an area of about 490 to 710 μm²)), polymorphic, with many exhibiting thick cytoplasmic projections (data not shown). BV2 cells in the presence of the treated yeast cells (i.e. yeast cells according to the present disclosure) are smaller than those in the presence of untreated yeast cells (“CTR” and “YP Glu 2%”). This is also the case for cells additionally treated with LPS (“LPS” and “YP Glu 2%+LPS”). Notably, under all tested conditions, the BV2 cells are smaller in the presence of treated yeast (i.e. yeast cells according to the present disclosure) than in the presence of untreated yeast. Taken together, the anti-inflammatory effects of the treated yeast cells (i.e. yeast cells according to the present disclosure) are evident.

### Patient Study

The effect of a composition according to the present disclosure on the symptoms of IBS and IBD was evaluated in a study. A dry composition according to the present disclosure was evaluated. The composition contained Saccharomyces carlsbergensis (deposited as DSM 34143), i.e. yeast cells treated as described herein, dried in sterile hot air and formulated as capsules (200 mg/capsule, which corresponds to about 5.8×10⁹ CFUs). Two groups of patients were treated. One group (“Group A”) received yeast cells (Saccharomyces cerevisiae (DSM 33148)) that had been treated as described above. The other group (“Group B”) received untreated yeast cells (Saccharomyces cerevisiae (DSM 33148)).

Group A included 6 subjects (6 females, 25-66 years old), out of which 5 were diagnosed with IBS and 1 was diagnosed with IBD.

Group B included 6 subjects (5 females, 39-70 years old, and 1 male, 79 years old), out of which 2 were diagnosed with IBS and 4 were diagnosed with IBD.

The subjects were instructed to take one capsule together with water before breakfast three times per week, i.e. a total of 600 mg/per week. After 12 weeks, each subject was asked to evaluate their symptoms of IBS/IBD and state whether they had experienced any improvement of the symptoms.

The study demonstrates that the symptoms of IBS and IBD were clearly improved by the treatment in all 6 subjects receiving Saccharomyces cerevisiae treated as described in the present disclosure (“Group A”). In the control group (“Group B”), receiving untreated yeast cells, only one subject perceived slightly less gas. Notably, only one subject experienced an adverse effect (nausea) and only one single time after intake. Otherwise, no adverse side effects were reported in the group receiving a composition according to the present disclosure. Individual subjects are described below. Each subject's comments are cited below following translation.

### Group a (Receiving a Composition According to the Present Disclosure)

Subject A1: Woman, age 66, diagnosed with IBS for 5 years. Symptoms: stomach pain, nausea, gas, diarrhea, constipation, bloating, heartburn and cramps. Perceived improvement: less stomach pain and bloating. Side effects: no adverse side effects.

Subject A2: Woman, age 56, diagnosed with IBS for 10 years. Symptoms: stomach pain, nausea, gas, diarrhea, constipation, bloating, heartburn, back-pain and cramps. Perceived improvement: less intestinal problems, stomach pain and heartburn. Side effects: no adverse side effects.

Subject A3: Woman, age 65, diagnosed with IBS for 22 years. Symptoms: Stomach pain, gas, diarrhea, and bloating. Perceived improvement: less bloating. Side effects: no adverse side effects.

Subject A4: Woman, age 25, diagnosed with IBS for 4 years. Symptoms: stomach pain, nausea, gas, diarrhea, bloating and heartburn. Perceived improvement: no stomach pain, no nausea, no heartburn, no diarrhea, only some gases and bloating left. Side effects: no adverse side effects.

Subject A5: Woman, age 28, diagnosed with IBD for 5 years. Symptoms: stomach pain, gas, diarrhea, constipation, bloating and cramps. Perceived improvement: faster recovery from relapses. Side effects: no adverse side effects.

Subject A6: Woman, age 51, diagnosed with IBS. Symptoms: stomach pain, nausea, gas, constipation and bloating. Perceived improvement: less constipation. Side effects: felt nauseous one time after intake; otherwise: no adverse side effects.

### Group B (Receiving Untreated Yeast)

Subject B1: Man, age 79, diagnosed with IBS for 6 years. Symptoms: stomach pain, nausea, gas, diarrhea, bloating and heartburn. Perceived improvement: none. Side effects: no adverse side effects.

Subject B2: Woman, age 50, diagnosed with IBD for 7 years. Symptoms: stomach pain, gas, diarrhea and bloating. Perceived improvement: none. Side effects: no adverse side effects.

Subject B3: Woman, age 70, diagnosed with IBD for 2 years. Symptoms: diarrhea. Perceived improvement: slightly less gas. Side effects: no adverse side effects.

Subject B4: Woman, age 39, diagnosed with IBD for 4 years. Symptoms: stomach pain, diarrhea, cramps and bloating. Perceived improvement: none. Side effects: no adverse side effects.

Subject B5: Woman, age 63, diagnosed with IBD for 42 years. Symptoms: stomach pain, nausea, gas, diarrhea and bloating. Perceived improvement: none. Side effects: no adverse side effects.

Subject B6: Woman, age 36, diagnosed with IBS for 8 years. Symptoms: stomach pain, gas, diarrhea, heartburn, cramps and bloating. Perceived improvement: none. Side effects: no adverse side effects.

1. A yeast cell for use in the treatment and/or alleviation of inflammation and/or symptoms caused by inflammation, wherein the yeast cell has been treated with electromagnetic waves in the range of 1 GHz to 300 GHz, or said yeast cell has been grown from a yeast cell treated with electromagnetic waves in the range of 1 GHz to 300 GHz.

2. The yeast cell for use according to claim 1, wherein the inflammation is selected from the group consisting of inflammatory bowel disease, irritable bowel syndrome; an autoimmune disease, and an inflammation caused by a microorganism and/or wherein the symptom caused by inflammation is chosen from the group consisting of heat, pain, redness, swelling, loss of function and combinations thereof.

3. The yeast cell for use according to claim 1, wherein the inflammation is inflammatory bowel disease, such as Crohn's disease or ulcerative colitis; or irritable bowel syndrome.

4. The yeast cell for use according to claim 1, wherein said electromagnetic waves are in the range from about 1 GHz to about 200 GHz, such as 10 to 100 GHz, such as 30 to 70 GHz, such as 40 to 65 GHz.

5. The yeast cell for use according to 4, wherein said electromagnetic waves are chosen from group consisting of: 30 GHz, 31 GHz, 32 GHz, 33 GHz, 34 GHz, 35 GHz, 36 GHz, 37 GHz, 37.5 GHz, 38 GHz, 39 GHz, 40 GHz, 41 GHz, 42 GHz, 43 GHz, 44 GHz, 45 GHz, 46 GHz, 47 GHz, 48 GHz, 49 GHz, 50 GHz, 51 GHz, 52 GHz, 53 GHz, 54 GHz, 55 GHz, 56 GHz, 57 GHz, 58 GHz, 59 GHz, 60 GHz, 61 GHz, 62 GHz, 63 GHz, 64 GHz, 65 GHz, 66 GHz, 67 GHz, 68 GHz, 69 GHz, 70 GHz, 71 GHz, 72 GHz, 73 GHz, 74 GHz, 75 GHz, 76 GHz, 77 GHz, 78 GHz, 79 GHz, 80 GHz, 81 GHz, 82 GHz, 83 GHz, 84 GHz, 85 GHz, 86 GHz, 87 GHz, 88 GHz, 89 GHz and 90 GHz.

6. The yeast cell for use according to claim 1, wherein said electromagnetic waves have a power density below 1 mW/cm²; preferably said electromagnetic waves have a power density of between 0.004 mW/cm² and 0.2 mW/cm².

7. The yeast cell for use according to claim 1, wherein said electromagnetic waves are modulated in a frequency within the range of 0.01% to about 0.5% of the average frequency.

8. The yeast cell for use according to claim 1, wherein said yeast cell has been treated with electromagnetic waves for a time period of from 10 minutes to 240 minutes, such as from 20 minutes to 130 minutes, such as from 20 minutes to 120 minutes, such as from 30 minutes to 90 minutes, such as from 35 minutes to 85 minutes, such as 45 minutes to 80 minutes, such as 40 minutes, such as 50 minutes, such as 60 minutes, such as 70 minutes, such as 80 minutes.

9. The yeast cell for use according to claim 1, wherein said yeast cell is of the genus Saccharomyces, such as a yeast cell being selected from the group consisting of Saccharomyces carlsbergensis or Saccharomyces cerevisiae.

10. A composition for use in the treatment and/or alleviation of inflammation and/or symptoms caused by inflammation, comprising at least one yeast cell according to any one of the previous claims and an excipient and/or a carrier.

11. The composition for use according to claim 10, wherein the composition further comprises at least one vitamin and/or at least one mineral.

12. The composition for use according to claim 10, wherein the composition is for oral intake.

13. The composition for use according to claim 10, wherein the composition is a liquid composition.

14. The composition for use according to claim 13, wherein the composition comprises the yeast cell in an amount of from 10×10⁶ CFUs/ml to 50×10⁶ CFUs/ml, preferably from 20×10⁶ CFUs/ml to 40×10⁶ CFUs/ml, such as 30×10⁶ CFUs/ml.

15. The composition for use according to claim 10, wherein the composition is a powder composition.
